(19) United States
(12) Patent Application Publication
(10) Pub. No.: US 2014/0309549 A1
(43) Pub. Date: Oct. 16, 2014

(54) METHODS FOR TESTING HEARING

(71) Applicant: Symphonic Audio Technologies Corp., San Francisco, CA (US)

(72) Inventors: Aaron Alexander Selig, San Francisco, CA (US); Varun Srinivasan, San Francisco, CA (US)

(73) Assignee: Symphonic Audio Technologies Corp., San Francisco, CA (US)

(21) Appl. No.: 14/178,034

(22) Filed: Feb. 11, 2014

Related U.S. Application Data
(60) Provisional application No. 61/763,163, filed on Feb. 11, 2013.
(60) Provisional application No. 61/831,796, filed on Jun. 6, 2013.
(60) Provisional application No. 61/867,436, filed on Aug. 19, 2013.
(60) Provisional application No. 61/880,367, filed on Sep. 20, 2013.

(51) Int. Cl. A61B 5/12

(52) U.S. Cl. CPC A61B 5/123
USPC 600/559

(57) ABSTRACT

One variation of a method for testing a hearing ability of a user includes: outputting a first audible tone including a first frequency; recording a first volume adjustment for the first audible tone by the user; outputting a second audible tone including a second frequency; recording a second volume adjustment for the second audible tone by the user; selecting a particular hearing model from a set of hearing models based on a difference between the first volume adjustment and the second volume adjustment, each hearing model in the set of hearing models including a hearing test result corresponding to a previous patient; and generating a hearing profile for the user based on the particular hearing model result.

### CROSS-REFERENCE TO RELATED APPLICATIONS

This application claims the benefit of U.S. Provisional Application No. 61/763,163, filed on 11 Feb. 2013, U.S. Provisional Application No. 61/831,796, filed on 6 Jun. 2013, U.S. Provisional Application No. 61/867,436, filed on 19 Aug. 2013, and U.S. Provisional Application No. 61/880,367, filed on 20 Sep. 2013, all of which are incorporated in their entireties by this reference.

### TECHNICAL FIELD

This invention relates generally to the field of hearing augmentation, and more specifically to a new and useful method for testing hearing in the field of hearing augmentation.

### BRIEF DESCRIPTION OF THE FIGURES

FIG. 1 is a flowchart representation of a first method in accordance with the invention;

FIG. 2 is a graphical representation of in accordance with one variation of the first method;

FIGS. 3A, 3B, and 3C are graphical representations in accordance with one variation of the first method;

FIG. 4 is a flowchart representation of one variation of the first method;

FIG. 5 is a flowchart representation of a second method in accordance with the invention; and

FIG. 6 is a flowchart representation of one variation of the second method.

### DESCRIPTION OF THE EMBODIMENTS

The following description of the embodiments of the invention is not intended to limit the invention to these embodiments, but rather to enable any person skilled in the art to make and use this invention.

### 1. First Method

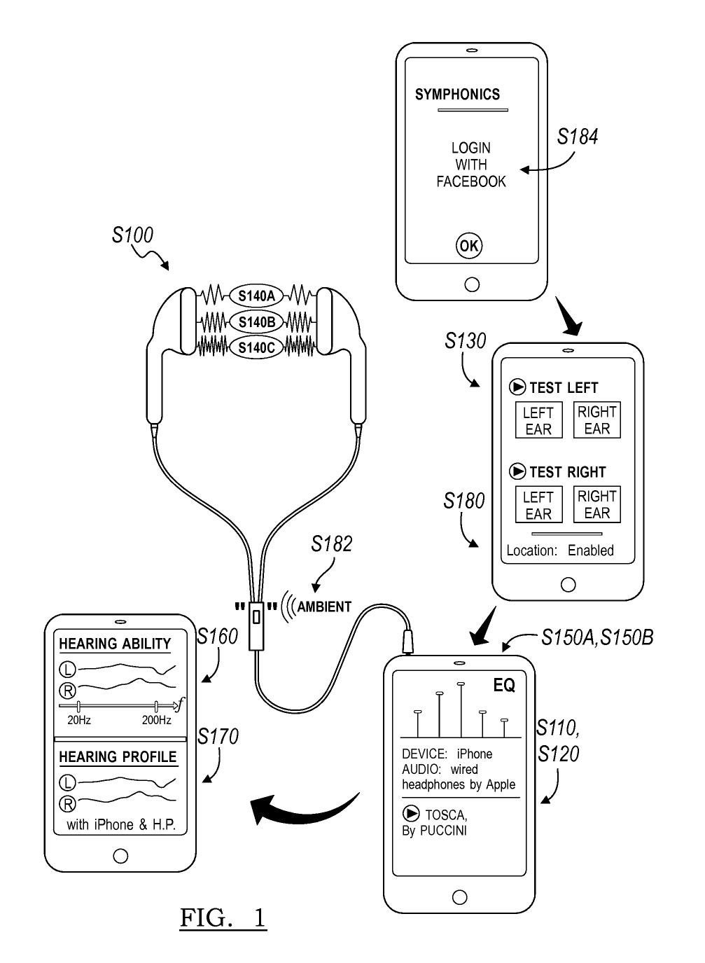

As shown in FIG. 1, a first method S100 for testing hearing includes: identifying an audio output device worn by a user in Block S110; accessing a sound profile for the audio output device in Block S120; pairing a channel of the audio output device with each of the left and right ears of the user based on a user poll in Block S130; for each of the left and right ears of the user, outputting a first signal with tones in the first hearing frequency band, the first signal of a first amplitude and including a first tone in Block S140A and recording a first user input in response to the first musical signal in Block S150A; for each of the left and right ears of the user, outputting a second signal within a second hearing frequency band different than the first hearing frequency band, the second musical signal of a second amplitude and including a second tone different than the first tone in Block S140B and recording a second user input in response to the second signal in Block S150B; estimating a hearing ability of each of the left and right ears of the user based on the first and second signals and the user inputs in Block S160; and generating a baseline hearing profile for the user according to the sound profile of the audio output device and the estimated hearing ability of each of the left and right ears of the user in Block S170.

The first method S100 functions to test a user's hearing with various tone- or music-based audio signals, wherein each audio signal is a unique tone or set of tones and is associated with a (distinct) hearing frequency band, in order to generate a baseline hearing profile of the user based on the results of the hearing test. Generally, the first method S100 outputs various sound signals, records a user's responses to those sound signals, and processes the user's responses in light of the musical sound signals to create a map of the user's hearing ability and/or to identify the user's hearing needs. This baseline hearing test implemented by the first method S100, can thus enable front-end calibration and/or generation of a user's hearing profile, which can be implemented in various scenarios to augment the user's hearing. In one example implementation, the hearing profile can be leveraged by an audiologist to customize a hearing aid for the user. In another example implementation, the hearing profile can be implemented by an application executing on a mobile computing device (e.g., a smartphone) to output augmented audio signals tailored to the user's hearing needs, such as in real time in response to a changing environment, changing environmental conditions proximal the user, or changing user hearing needs.

The first method S100 can be implemented by one or more computer systems, such as a cloud-based computer system (e.g., Amazon EC2), a mainframe computer system, a grid-computer system, or any other suitable computer system. For example, the first method S100 can be implemented by a native application executing on a mobile computing device, such as a smartphone, a tablet, or a peripheral audio device. In another example, the first method S100 can be implemented through software executing on embedded circuitry incorporated into an audio output device, such as a speaker, a headphone, or a Bluetooth headset. In another example, the first method S100 can be implemented by a remote server in cooperation with a native application executing on a mobile computing device. The computer system can also incorporate a user interface through which the user can input responses to signals, review a hearing profile, enter demographic or other personal information, upload music or audio files, or enter, access, or review any other data or information. The user interface can be accessible through a web browser or through a native application executing on a (mobile) computing device, such as a laptop computer, a desktop computer, a tablet, a smartphone, a personal data assistant (PDA), a personal music player, etc. Generally, the audio output device can include any device that outputs a sound, such as a headphone, a speaker, or a mobile phone. The computing device can include any device that processes a digital signal, such as a headset incorporating a microprocessor, a smartphone, or a tablet. However, the audio output device and computing device can be any other suitable type of device and can be discrete and/or physical coextensive (i.e., embodied in the same device).

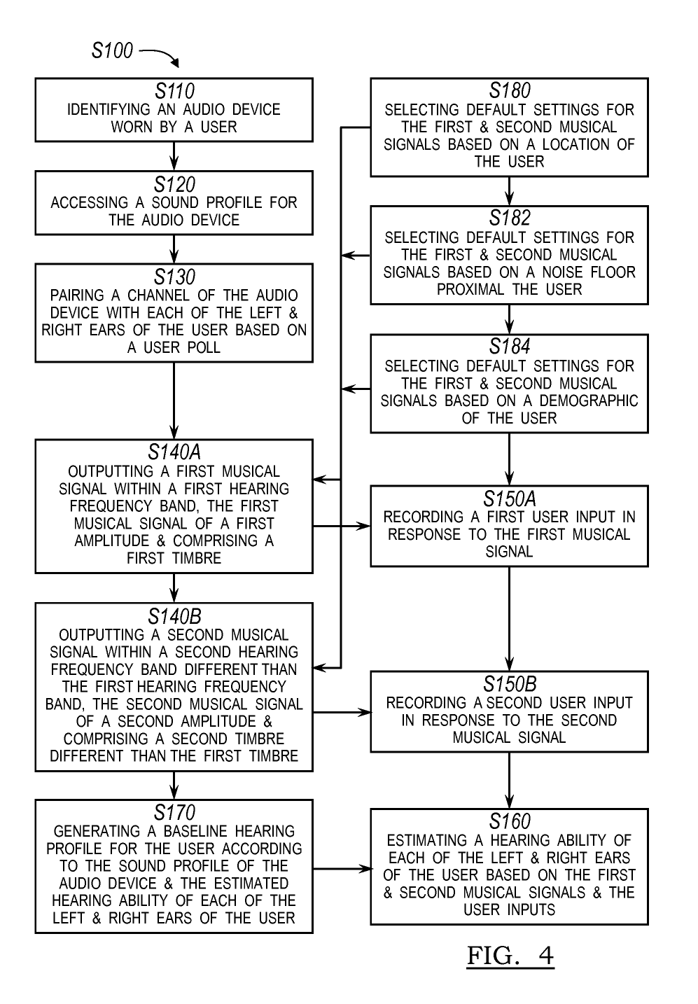

As shown in FIGS. 1 and 4, Block S110 of the first method S100 recites identifying an audio output device worn or in use by a user. Generally, Block S110 functions to identify one or more hardware components that cooperate to output the signal as sound to the user such that Block S120 can select a sound profile for each of the one or more hardware components and such that Block S170 can generate the baseline user hearing profile that is substantially hardware-independent. Block S110 can identify an audio output device, such as a headset, a set of in-ear headphones, a set of over-ear headphones, an external speaker, or other audio output device incorporating a speaker, through which Blocks S140A and S140B output signals to the user. Block S110 can additionally or alternatively identify a computing device coupled to the audio output device, such as a smartphone, tablet, MP3 player, laptop computer, or desktop computer. The audio output device can be connected to the computing device wirelessly or through a wired connection. The audio output device can alternatively incorporate the computing device.

In one example implementation, Block S110 identifies the audio output device and/or the computing device by prompting the user to select an audio output device model and/or a computing device model from a drop-down menu within a user interface on the computing device. In another example implementation, Block S110 identifies the audio output device and/or the computing device by accessing an internal serial number or product number stored digitally on the audio output device and/or computing device. For example, Block S110 can poll the audio output device that is a wireless (e.g., Bluetooth headset) for a serial or product number of the audio output device. Similarly, Block S110 can identify the computing device by accessing an internal digital product or serial number and then select an audio output device (i.e., a device that outputs audible signals) that is commonly paired with the identified computing device. For example, if Block S110 identifies the computing device as a particular model of phone of a particular generation and by a particular manufacturer, Block S110 can estimate that the audio output device is a stock set of in-ear headphones sold with that particular generation of phone.

In another implementation, Block S110 can identify the computing device and then retrieve a corresponding audio output characteristic from a database or from the computing device itself. For example, Block S110 can detect a resistance across an input jack, plug, or other connector of the audio output device and then match the detected resistance to a stored resistance value associated with a known audio output device profile to predict the audio output device connected to the computing device. In a similar example, Block S110 can detect the resistance across the input jack of the audio output device and adjust the strength (i.e., amplitude) of the output signals accordingly.

However, Block S110 can function in any other way to identify an audio output device worn by a user and/or a computing device used by the user during a hearing test.

As shown in FIGS. 1 and 4, Block S120 of the first method S100 recites accessing a sound profile for the audio output device. Because audio output response can vary widely for speakers of different sizes, materials, wind densities, core geometries, cone geometries, shielding, wire size, wire geometry, housing materials, housing thickness, housing geometry, etc., Block S120 can function to source a profile for the audio output response of the audio output device such that Blocks S140A and S140B can account for the output response of the audio output device and/or such that Block S170 can filter out the audio output response of the particular audio output device used by the user during the hearing test to generate a substantially audio output device-independent baseline hearing profile of the user. Because audio amplifiers can exhibit similarly varying audio signal output responses, Block S120 can further source an audio signal output response of the computing device and combine this with the audio output response of the audio output device such that Block S170 can generate the baseline hearing profile of the user that is both audio output device- and computing device-agnostic. In one example implementation, Block S120 communicates with a remote database that contains audio response profiles of one or more audio output devices, one or more computing devices, and/or one or more combinations of an audio output device coupled to a computing device. In this example implementation, Block S120 can select a particular sound profile from the list of profile stored on the remote database. Block S120 can similarly select a sound profiles from a local database.

In another example implementation, Block S120 can generate the sound profile for the audio and computing device substantially in real time. For example, Block S120 can prompt the user to set a microphone beside a speaker of the audio output device, wherein Block S120 outputs a drive signal to the speaker and records the audio output of the speaker via the microphone. Block S120 can subsequently compare the drive signal to the recorded audio output to extract an output response of the combined microphone, computing device, and audio output device. For the microphone that is incorporated into the computing device (or audio output device), Block S110 can identify the computing device (or audio output device) as described above, Block S120 source an output response profile of the microphone according to the identified computing device, and Block S120 can further filter the output response from the microphone from the output response of the combined microphone, computing device, and audio output device based on an output response profile of the microphone. Block S120 can thus isolate an output response of the combined computing device and audio output device that can be filtered implemented in Block S170. This can be particularly useful for substantially uncommon or less common audio output devices, computing devices, and/or combinations of audio output devices and computing devices. However, Block S120 can function in any other way to access a sound profile for the audio output device, computing device, amplifier, and/or other devices connected to the audio output device.

As shown in FIGS. 1 and 4, Block S130 of the first method S100 recites pairing a channel of the audio output device with each of the left and right ears of the user based on a user poll. For a stereo audio output device, Block S130 functions to identify which speaker of the audio output device is in, on, or proximal the left each of the user and which speaker of the audio output device is in, on, or proximal the right each of the user. For a mono-channel audio output device, Block S130 functions to identify which ear of the user is engaged with or substantially proximal the speaker of the audio output device. Because the user may inadvertently or purposefully place a left-channel headphone in his right ear and vice versa, Block S130 can substantially prevent Block S140A from inadvertently outputting a signal meant for the user's left ear to the user's right ear by identifying the placement of each speaker of the audio output device prior to testing in Blocks S140A, S140B, S150A, and S150B. Block S130 can thus enable the first method S100 to identify each ear and to generate a user baseline hearing profile that includes unique hearing profiles for each ear.

In one example implementation, Block S130 can output a first auditory signal through a first speaker of the audio output device and prompt the user to select whether the signal was heard in his left ear or his right ear. For example, in response to outputting a first auditory signal, Block S130 can prompt the user to select a “LEFT” button or a “RIGHT” button from within a user interface of a native application executing on the computing device that is a smartphone with a touchscreen. In this example implementation, Block S130 can further confirm the user's selection by outputting a second auditory signal through a second speaker of the audio output device and again prompting the user to select whether the signal was heard in his left ear or his right ear. However, Block S130 can pair a channel of the audio output device with each of the left and right ears of the user in any other way or according to any other schema.

As shown in FIGS. 1 and 4, Block S140A of the first method S100 recites outputting a first signal within a first hearing frequency band, the first signal of a first amplitude and including a first tone or set of tones. Similarly, Block S140B of the first method S100 recites outputting a second signal within a second hearing frequency band different than the first hearing frequency band, the second signal of a second amplitude and including a second tone or set of tones different than the first tone or set of tones. Generally, the first method S100 can implement Blocks S140A and S140B for each (i.e., left and right) ear of the user. However, the first method S100 can implement Blocks S140A and S140B for a single ear of the user, such as the user's dominant ear or for an ear in which the user commonly wears a single-ear headset or a single earbud. For example, for the audio output device that is a stereo output device incorporating two speakers, the first method S100 can first implement Blocks S140A and S140B for the user's first (e.g., left) ear and, following completion of Blocks S140A and S140B for the first ear, repeat Blocks S140A and S140B for the user's second (e.g., right) ear. Alternatively, the first method S100 can implement Blocks S140A and S140B simultaneously for each ear or for a single ear, such as for the audio output device that is a mono-channel audio output device. The first method S100 can also implement Blocks S140A and S140B in oscillation, wherein the first method S100 alternates between S140A and S140B to switch back and forth from the user's left ear to the user's right ear for each frequency band.

Each of Blocks S140A and S140B function to output signals of a unique tone or set of tones within the set of signals output during the hearing test. Generally, each signal can include a non-zero amplitude over a continuous band of frequencies within the hearing frequency band of the respective signal. Each signal therefore can include a range of frequencies rather than only a single frequency. Furthermore, each signal can be a continuous audio file including varying amplitudes of various frequencies within the respective hearing frequency band over time.

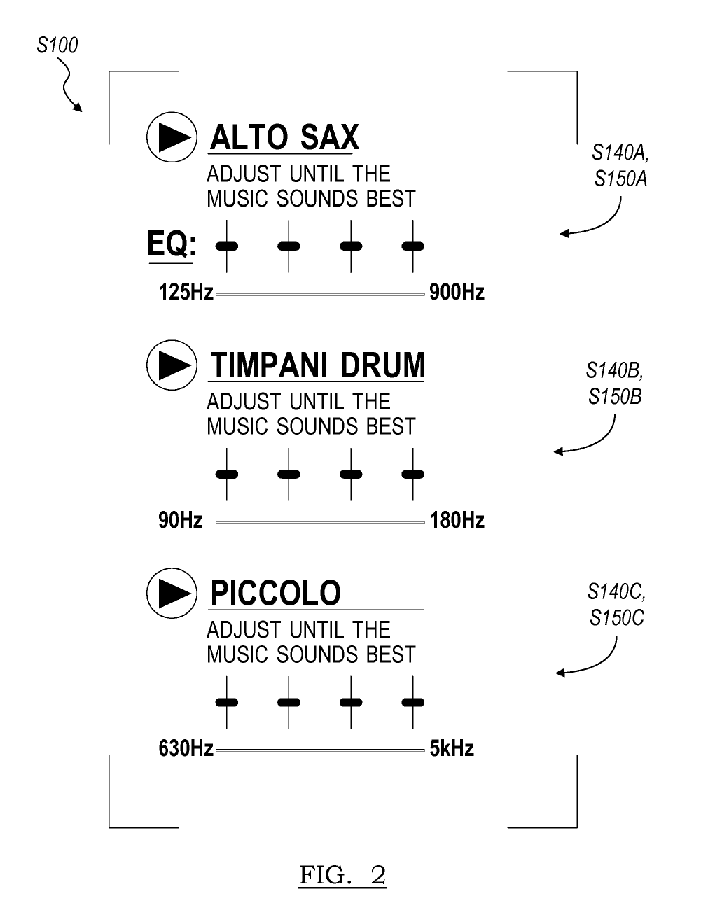

In one implementation, each signal includes a musical riff played by one (or more) particular type of instrument. In one example, the first method S100 can include outputting a first signal of an alto saxophone in Block S140A, outputting a second signal of a baritone saxophone in Block S140B, and outputting a third signal of a tenor saxophone in Block S140C. In another example shown in FIG. 2, the first method S100 can include outputting a first signal of an alto saxophone in Block S140A, outputting a second signal of a Timpani drum in Block S140B, and outputting a third signal of a piccolo in Block S140C. In yet another example, the first method S100 can include outputting a first signal of a trumpet and flute in Block S140A, outputting a second signal of a cello and an oboe in Block S140B, and outputting a third signal of a tuba and ⅞ upright bass in Block S140C. In this implementation, each signal can include multiple different notes played be each respective instrument such that multiple hearing frequencies, within the hearing band of the respective signal, are tested with the same signal. Each signal can additionally or alternatively include a series of chords, a solo, or any other set of musical notes within a single musical key or set of musical keys.

In another implementation, each signal includes a song or a portion of a song with sounds that fall substantially within a respective hearing frequency band of the particular signal. In one example, the first method S100 can include outputting a portion of a recorded solo by Maria Callas (soprano) performing in La Scala by Puccini in Block S140A, outputting a portion of a recorded solo by Arigo Pola (tenor) performing in Tosca by Puccini in Block S140B, and outputting a portion of a recorded solo by Feodor Chaliapin (bass) performing in The Maid of Pskov by Rimsky-Korsakov in Block S140C. In another example, the first method S100 can include outputting a first signal that is a several-second, high-frequency portion (e.g., fast guitar solo) of a recording of All Along the Watchtower by Jimmy Hendrix in Block S140A, outputting a second signal that is a verse (e.g., midrange frequency portion) of the recording of All Along the Watchtower by Jimmy Hendrix in Block S140B, and outputting a third signal that is a several-second, low-frequency portion (e.g., slow guitar solo) of the recording of All Along the Watchtower by Jimmy Hendrix in Block S140C.

The signals implemented in Blocks S140A and S140B can include wholly synthesized musical or tonal sounds, recordings of real instruments, or a combination of synthesized and authentic sounds. In the foregoing implementation in which the musical sounds are recordings, the first method S100 can cut or compressed recordings to a certain length, filter the recordings to remove frequencies outside of a selected hearing band frequency, or otherwise edit or manipulate the recordings to fulfill a particular signal requirement. The musical sounds that are recordings can be preset, such as predefined for a particular hearing test, or can be sourced from the user's personal media or from an external media database. For example, the first method S100 can source a song from a music library (e.g., iTunes) stored on the computing device that implements the first method S100, analyze the song to determine if or what parts of the song are appropriate to test user hearing with a particular hearing frequency band, and filter, cut, edit, or otherwise manipulate the song to prepare the song for its use in a hearing test. The signals can also be sourced from a remote media account of the user (e.g., iCloud) or from a third-party media provider (e.g., Amazon, Pandora).

Alternatively, Block S140A and S140B can output signals that are other than or include other than signals. For example, Blocks S140A and/or S140B can output a recording of a sporting event or broadcast (e.g., car race, baseball game), a spoken voice, a speech, a lecture, a conversation, an interview, or any other event with sound within a certain frequency range. However, the signals can be of any other type, content, length, or frequency band. Each signal can also be a pure tone. Furthermore, the first method S100 can include outputting additional musical or auditory signals, such as for different hearing frequency bands and/or to verify a user response to a previous test with a different musical or auditory signal. Block S140 can also implement portions of an otoacoustic emissions (OAE) test, such as by transmitting an audio signal through a plug inserted into an ear of the user, wherein a microphone within the plug records otoacoustic emissions of the normal ear in reaction to the audio signal. Similarly, Block S140 can implement portions of an auditory brainstem response (ABR) test, such as by transmitting an audio signal through a plug inserted into an ear of the user, wherein electrodes attached to the user's scalp measure the user's brain activity in response to the audio signal. Therefore, the first method S100 can test any number of frequency bands within the audible range for humans with musical and/or other auditory signals with discrete and/or overlapping frequencies.

Block S140A and S140B can also output visual signals to indicate to the user that an audio signal (e.g., a signal) is currently output by the audio output device. In one implementation, Block S140A displays, on the user interface executing on the computing device, a visual indicator of an audio signal communicated to the audio output device and outputted as sound. In one example, Block S140A can alter the color of a region displayed within the user interface, such as from blue to green, to indicate that sound is currently output from the audio output device. Block S140A can additionally or alternatively modify a size or shape of a visual indicator displayed within the user interface. For example, Block S140A can enlarge a displayed circle or set of circles rendered on the display, such as according to a magnitude of an output audio signal. In another example, Block S140A can display a visual waveform of the output audio signal. Block S140B can implement similar functionality. Therefore, Block S140A and Block S140B can output a visual representation corresponding to an output audio signal, thereby enabling the user to visually discern that an audio signal is in playback even if the user cannot audibly discern (i.e., hear) the audio signal through the audio output device.

As shown in FIGS. 1 and 4, Block S150A of the first method S100 recites recording a first user input in response to the first signal. Similarly, Block S150B of the first method S100 recites recording a second user input in response to the second signal. Generally, Blocks S150A and S150B function to capture user responses to signals played for the user in Block S140A and S140B, respectively. Block S150A and S150B can prompt the user to respond to played sound by inputting responses through a user interface within a native application executing on a computing device (e.g., smartphone) that implements the first method S100. For example, the user can respond to a signal through a touchscreen incorporated into the computing device. Block S150A and S150B can alternatively prompt the user to respond to played sound by shaking, tilting, or otherwise manipulating computing device (e.g., tablet) that implements the first method S100. Block S150A and S150B can alternatively prompt the user to respond to played sound by talking into a microphone coupled to the computing device, by providing an input into an external keyboard, mouse, or other external device, or in by responding to the musical sound in any other suitable way.

In one example implementation, Blocks S140A, S140B, etc. can cooperate to output first signals that include a recorded voice (or voices) speaking numbers (e.g., 1, 8, 2, 5, 9), wherein each audio signal corresponds to a particular spoken number and covers a different frequency band or exhibits a different dominant frequency amongst the set of spoken numbers. As Block S140A, S140B, etc. audibly output signals, Blocks S150A, S150B, etc. can simultaneously and correspondingly prompt the user to select a number from a keypad—displayed on a touchscreen of the computing device—that matches each most-recently spoken number. In this example implementation, the first method S100 thus prompts the user to enter what he thinks he heard, which can offer greater resolution to the user's hearing ability than an input that is simply one of ‘yes I heard the sound’ or ‘no I did not hear the sound.’

In another example implementation, Block S140A can output a signal that includes a song recording with components outside a desired frequency range filtered out, and Block S150A can prompt the user to adjust an audio engine parameter (e.g., an equalizer setting) for the recording, in real time as the signal is played, until the user finds a preferred or ‘best’ audio engine parameter, such as shown in FIG. 2. In this example implementation, Block S160 can correlate a user's decrease in signal amplitude at a first frequency range as a good or hearing in that frequency range, an unchanged EQ setting at a second frequency as adequate hearing in that frequency range, and an increase in signal amplitude at a third frequency range as poor hearing that that frequency range. Alternatively, Block S150A can prompt the user to adjust an audio engine parameter up to a point at which the user can just barely hear the signal and/or adjust an audio engine parameter down to a point at which the user can no longer hear the signal.

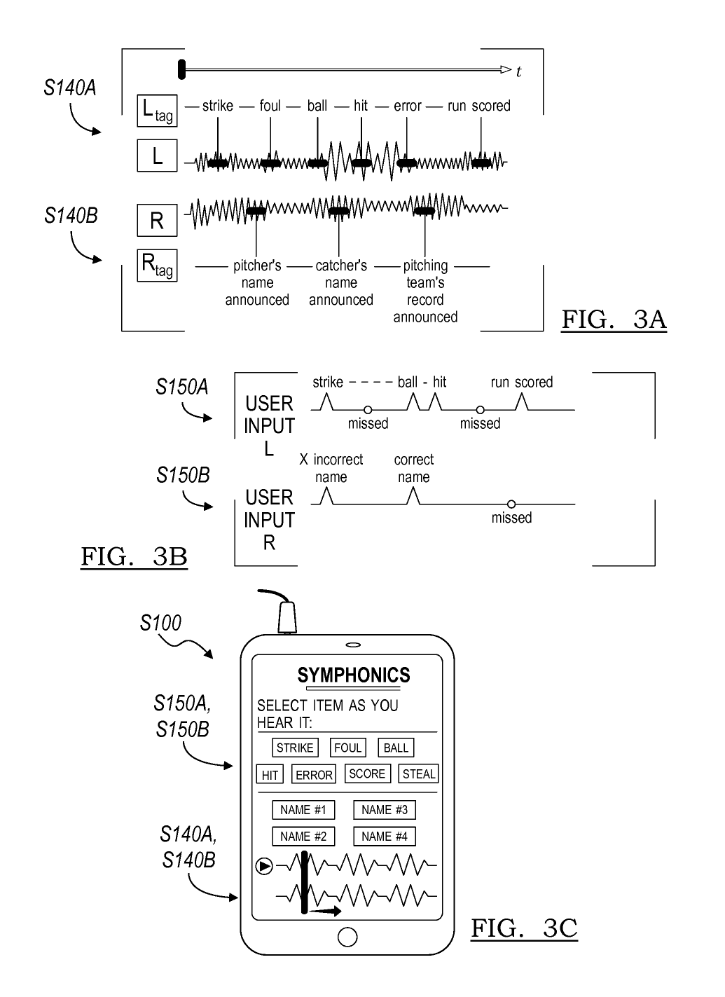

In yet another example implementation, Blocks S140A, S140B, S140C, etc. can cooperate to output a signal that includes a portion of a recording of a sporting event, wherein certain events or announcements within the event, which correlate with sounds within certain frequency ranges, are associated with one of Blocks S140A, S140B, or S140C, etc. As the user listens to the signal, Block S150A can prompt the user to serially identify particular events as then occur within the sporting event by selecting from various labeled input regions. In one example, the sporting event is a baseball game, as shown in FIGS. 3A, 3B, and 3C. In this example, the signal can be tagged with the particular events, as shown in FIG. 3B. Furthermore, input regions, labeled with “strike,” “foul,” “ball,” “hit,” “error,” “score,” “steal,” etc. are displayed within the user interface, such as on a touchscreen of the computing device implementing the first method S100, as shown in FIG. 3C. In this example, Block S160 can estimate the user's hearing ability based on events that the user misses entirely, events that the user labels incorrectly, and/or how quickly the user responds to an event (shown in FIG. 3B).

In a further example implementation, each of Blocks S140A, S140B, S140C, etc. can output a signal that is a gaming sound in a unique frequency range over the audible range, and Blocks S150A, S150B, S150C, etc. can thus prompt the user to respond to the unique signals in a gaming environment. For example, Blocks S150A, S150B, S150C, etc. can prompt the user to perform a particular game-type action in response to a particular musical sound and can reward the user with a set of points given correct and/or timely responses. However, Blocks S140A and S140B can output any other signals, and Block S150A and S150B can prompt the user to respond to the signals in any other suitable way. Furthermore, as described above, Blocks S140A, S140B, etc. and S150A, S150B, etc. can test a single ear of both ears of the user, either simultaneously or serially.

In another implementation, Blocks S150A, S150B, S150C, etc. prompt the user to compare two tones or sets of tones from a first frequency band and to choose which tone or set of tones is preferred. Blocks S150A, S150B, S150C, etc. can repeat these prompts until the first method S100, application, and/or tester determines that adequate user preferences for a tested frequency band have been collected. The first method S100 can repeat these steps for each tested frequency band to generate a map of the user's hearing profile.

The foregoing example implementations can additionally or alternatively verify calibration settings of a previous test. Previous tests can be imported or entered manually into the software by any suitable means. Similarly, this and other example implementations can be used to tailor a subsequent hearing test, such as to select key frequency bands to test rather than the full hearing spectrum of the user.

Though potentially less scientific than current hearing tests that use buzzers and/or single-frequency sounds to test hearing, Blocks S140A, S140B, etc. and S150A, S150B, etc. can enable ball-park testing of user hearing ability in a substantially experience-driven environment without specialized testing equipment and without an audiologist. Furthermore, because Blocks S140A, S140B, etc. and S150A, S150B, etc. can enable a comfortable and interesting experience-driven testing environment that can be implemented on a user's personal computing device (e.g., smartphone), the first method S100 can reduce barriers to user hearing tests and thus lead to more user hearing tests over time, which can better enable the first method S100 to track user hearing changes over time than annual or biannual hearing tests.

As shown in FIGS. 1 and 4, one variation of the first method S100 includes Block S180, which recites selecting default settings for the first and second signals based on a location of the user. For example, Block S180 can access GPS data from the computing device implementing the first method S100 to determine the location of the user, such as down to the state, city, block, nearest intersection, building, floor of the building, or room of the building, etc. in which the user is located. Generally, Block S180 functions to resolve the location of the user to a suitable resolution in order to leverage hearing test results of other users in same room or similar location. Additionally or alternatively, Block S180 can select a particular type of test and/or enable unique hearing profiles (e.g., home, work, gym) of the user according to the location of the user.

In one example implementation, the first method S100 implements hearing test results of other users in the same or similar location of the user to select a starting point for a new user hearing test for the user, whether a new user or an existing user in a new location. Based on the users locations, Block S180 can access baseline hearing profiles of other users in the same or similar locations by extracting trends or similarities across other baseline hearing profiles of other users and adjusting default signal settings according to the trends or similarities. For example, Block S180 can implement location-based hearing test trends to select suitable musical sound types or initial amplitude or audio engine parameters for musical outputs for the location. In another example, Block S180 can determine that the user is in a particular room and identify trends in other user hearing tests, performed in the same or similar room, that include increased ability to hear in the 20-200 Hz range and decreased ability to hear in the 1000-2000 Hz range. Block S180 can thus correlate the former with local resonance in the 20-200 Hz range within the room, and Block S180 can correlate the latter with sound absorption and attenuation within the 1000-2000 Hz range. Block S140A, S140B, etc. can thus implement these correlations by decreasing the amplitude of a signal in the 20-200 Hz range and increasing the amplitude of a signal in the 1000-2000 Hz range. However, Block S180 and the first method S100 can access and implement user location data in any other suitable way.

As shown in FIGS. 1 and 4, one variation of the first method S100 further includes Block S182, which recites selecting default settings for the first and second signals based on a noise floor proximal the user. Generally, Block S182 tests a noise floor proximal the user and adjust signal settings to compensate for the noise floor. For example, Block S182 can record ambient noise conditions through a microphone incorporated into a headset or headphones worn by the user or into the computing device implementing the first method S100. Block S180 can analyze the ambient noise recording (e.g., via Fourier analysis) and compensate for ambient noise at certain frequencies and/or levels by adjust thing the signals output in Blocks S140A, S140B, etc. However, Block S182 and the first method S100 can determine ambient noise conditions and adjust signal setting and/or testing parameters in any other suitable way.

As shown in FIGS. 1 and 4, one variation of the first method S100 further includes Block S184, which recites selecting default settings for the first and second signals based on a demographic of the user. For example, Block S184 can extract the age of the user from a social networking system, access hearing test results of other users of similar age, and adjust hearing test parameters (e.g., audio engine parameters or frequency bands tested) according to trends in hearing tests of the other users. In another example, Block S184 can access hearing data and/or hearing test results of relatives of the user, as identified in a social networking system, to determine a genetic propensity for hearing ability of disability for the user. However, Block S184 and the first method S100 can function in any other way to collect and implement personal user data to improve hearing test accuracy and/or the user's hearing test experience.

The first method S100 can also include selecting default settings for the first and second signals based on past user hearing data. Generally, the first method S100 can apply past user hearing data to default settings of the hearing test of the first method S100 in order to reduce the time required to achieve adequate test results under the first method S100. In one example, the first method S100 can access results of an OAE test or an ABR test, as described in Block S140 above, and adjust the default settings of the test based on hearing ability or inability of the user as suggested by the OAE or ABR test. In another example, the first method S100 can collect recent user hearing data from the user's medical records, such as a current hearing aid prescription and/or hearing aid tuning settings entered by an audiologist. Therefore, the first method S100 can customize the user's hearing test according to past user hearing data to improve the efficiency of the hearing test.

Prior to estimating the hearing ability of the user in Block S160, the first method S100 can repeat Blocks S140A, S140B, etc. and S150A, S150B, etc. to verify user responses to the signals. In implementing the foregoing Blocks for a second, third, or fourth time, etc., the first method S100 can repeat the same signals or different signals, adjust signal parameters, adjust a testing scenario (e.g., hearing test game), etc. for the subsequent tests. The first method S100 can also implement Blocks S140A, S140B, etc. and S150A, S150B, etc. under the guidance of a second entity, such as an audiologist engaging in a phone call with the user during the hearing test or an automaton executing on a remote server and implementing voice recognition and voice menus to guide the user through the hearing test. Blocks of the first method S100 can therefore be implemented across multiple devices and/or interfaces substantially simultaneously to generate an “earprint” of the user's hearing ability. For example, an audiologist can step through the test on a tablet computer while the patient responds to audio signals played back on a mobile phone. In this example, the audiologist and the user can occupy the same space (e.g., a physician's office), or the audiologist and the user can be remotely located, such as in different buildings, in different cities, or in different countries.

As shown in FIGS. 1 and 4, Block S160 of the first method S100 recites estimating a hearing ability of each of the left and right ears of the user based on the first and second signals and the user inputs. Because each signal of Blocks S140A, S140B, etc. is associated with a particular hearing frequency band, users responses to the each signal can be indicative of how well the user can hear each frequency band. Therefore, Block S160 can analyze and combine user responses to the multiple signals to identify user hearing ability across the audible range. For example, Block S160 can associate an amplitude adjustment over a whole signal with user hearing ability over whole band frequency of the signal. In another example, Block S160 can identify user hearing ability at various frequencies within the hearing frequency band of the signal based on user audio engine parameter adjustments during playback of the signal. In a further example, for hearing tests that include games or that prompt the user to respond to content within a signal can estimate user hearing ability based on the occurrence of a user response, the timing of a user response, and/or the content or type or user response.

Generally, Block S160 can estimate user hearing ability in each hearing frequency band associated with each signal output in Blocks S140A, S140B, S140C, etc. The first method S100 can output multiple signals to test multiple hearing frequency bands across the audible range, wherein each additional signal beyond the first and second signal increases the resolution of the estimated hearing ability of the user. Therefore, Block S160 can estimate user hearing ability in one hearing frequency band based on a user response to one signal and combine this estimate with estimates in other hearing bands to generate a holistic estimate of user hearing ability across the tested audible band. Block S160 can estimate user hearing ability in each ear separately, both ears on combination, and/or a single ear.

Block S160 can also adjust a granularity of user controls during and/or after the hearing test based on an early input by the user before or during the hearing test. For example, based feedback entered by the user after playback of a first section of a tone, Block S160 can provide additional (or modify existing) feedback controls to (or for) the user. For example, once the user provides inputs indicating that he can hear very soft tones during a current hearing test, Block S160 can modify user feedback controls to enable the user to provide finer-grained feedback in response to a subsequent tone. Block S160 can similarly cooperate with Blocks S140A, S140B, S150A, and/or S150B, etc. to adapt the hearing test for the user. For example, Block S140A can test a first tone on the user's left and right ears, and Block S160 can determine—based on user feedback to the first tone captured in Block S150A—whether to test the second tone output in Block S140B both of the user's ears or to perform separate hearings tests on the user's left ear and the user's right ear. However, Block S160 can function in any other way to estimate the user's hearing ability.

As shown in FIGS. 1 and 4, Block S170 of the first method S100 recites generating a baseline hearing profile for the user according to the sound profile of the audio output device and the estimated hearing ability of each of the left and right ears of the user. Generally, Block S170 combines the estimated hearing ability of the user, a previous estimated user hearing ability (e.g., from a previous test in the same or different location), the audio output device profile, the computing device profile, user location, local noise floor, user demographic, user inputs, and/or any other relevant information or data to generate an image of the user's hearing ability in the form of a hearing profile. The hearing profile can indicate which frequencies the user hears well, which frequencies the user does not hear well, how much gain in each frequency band a user needs to compensate for hearing loss, which ear the user favors, environments or scenarios in which the user has difficulty hearing, frequencies that the user wishes to augment, frequencies that the user wishes to attenuate, or any other hearing-related parameter of the user.

In one example implementation, the hearing profile can be used to set parameters for a custom hearing aid for the user. Therefore, the hearing profile can thus be sent or otherwise made available to an audiologist such that the audiologist can program a hearing aid for the user.

In another example implementation, the hearing profile can be implemented as an augmented hearing application that executes on a smartphone, tablet or other mobile device of the user, wherein the hearing application receives local sounds through a microphone incorporated in the mobile device, handset, or headphones, adjusts the local sounds according to the user's hearing profile, and outputs the adjusted local sounds through the headset or headphones worn by the user to augment the user's hearing experience. For example, the hearing profile can be implemented to aid the user in face to face conversation with a single person or small group of people in a closed room, to aid the user in face to face conversation with multiple people in a crowded and boisterous space (e.g., while at dinner in a crowded restaurant), to aid the user in hearing a speech or lecture in an auditorium, or to aid the user in holding a phone conversation with a person several miles or thousands of miles away.

The hearing profile can also be shared across and implemented by multiple other devices, such as an audio tour system, multiple electronic devices used by the user (e.g., a smartphone, a tablet, a laptop computer, and a hearing aid), or devices shared across friends, family, coworkers, etc. However, the hearing profile generated in Block S170 can be implemented in any other way, by any other device, and in any other suitable scenario.

The first method S100 can additionally or alternatively leverage input data, such as user profile data captured through user input or collected from social networks or other data sources, to trigger a reminder or a prompt to perform a hearing test. In one example implementation, the first method S100 leverages the user's age to define how often the user should perform the hearing test described above and prompts the user to take the test after a certain period has passed since a previous hearing test. In another example implementation, the first method S100 leverages location (e.g., GPS, IP address) to prompt or suggest that the user perform a hearing test at a new location. In yet another example implementation, the first method S100 detects a new sound environment, such as music playing in an auditorium or a quiet room, and prompts the user to test his hearing in the new sound environment. In another example implementation, the first method S100 prompts the user to perform a set of hearing tests at a predefined interval and/or at random times to collect a broad set of hearing test data, such as in the event that a medical or hearing professional requires a broad set of audio or hearing data on a patient in a variety of settings in order to customize a hearing aid to the user's needs.

As described above, the first method S100 can be implemented within a native application executing on a mobile computing device (e.g., a smartphone) to provide personal mobile access to a hearing test. The first method S100 can be similarly implemented on a computer network and accessed through a native application and/or web browser executing on a mobile computing device. However, engagement within native applications on mobile devices is often a function of user setup time and time to meaningful output, with time to meaningful output and level of engagement often inversely proportional. Therefore, the first method S100 can function to complete the hearing test within a limited target time, such as within forty-five seconds from start to finish or within sixty seconds of when a user first opens the native application. Within the limited target time, the first method S100 can thus collect baseline user hearing data, such as general hearing ability within three audible ranges. For example, the first method S100 can play five different audible tones in sequence in each of the low, mid, and high audible ranges and prompt the user to increase the volume of the output tones until the user can hear or distinguish each tone. However, though the results of this short hearing test may provide adequate baseline hearing data for the user, the test may neglect higher-resolution hearing data, such as the user's hearing ability within narrow audible ranges or frequency bands, specific audible frequencies that are difficult for the user to hear, hearing sensitivities of the user, etc. Therefore, results of a hearing test for a user may be characterized as data-sparse. Furthermore, user hearing information extracted from the test may be subject to errors and inaccuracies not immediately correctable due to lack of detailed hearing data from the hearing test. For example, ambient noise, local environment, a sound output profile of headphones, a speaker, and/or a smartphone user during the test, the user's attentiveness, etc. can all affect results (i.e., decrease the accuracy) of the hearing test.

However, as shown in FIG. 4, one variation of the first method S100 further includes Block S186, which recites interjecting additional hearing test results into the user's hearing test based on hearing test data of other users. Generally, Block S186 can implement pattern matching, machine learning, and/or other techniques to compare information of the user (e.g., hearing test data, demographic data) to information of other users, to extract data from the information of the other users and missing from the user's hearing test results, and to insert the extracted data into the user's hearing test results.

In one implementation, Block S186 compares the user's hearing test results to stored hearing test results of other users based on a demographic similarity between the user and the other users associated with stored test results. In one example, the first method S100 prompts the user to enter demographic information into the native application directly, such as age, gender, height, weight, personal medical history or events, family medical history, occupation, etc. In another example, the first method S100 prompts the user to log into an online social network and then extracts relevant user demographic information directly from the online social network. The first method S100 can additionally or alternatively interface with a location (e.g., GPS) sensor within the mobile computing device to determine the location of the user during the hearing test and then retrieve location-specific information based on a time of day during which the user takes the test, such as common ambient noises, crowds, and/or local wind or weather patterns. The first method S100 can similarly interface with a connected microphone to detect ambient noises, noise level, or other local distractions related to or indicated by sound. Block S186 can thus access one or more user demographic, local noise, and/or location data for the current hearing test and compare these data to hearing test results stored in the database, any of which may be tagged with similar demographic, local noise, and/or location data. Once Block S186 selects a stored hearing test (or a stored hearing test model based on multiple hearing tests from multiple users and) pertinent to the current user, Block S186 can extract relevant data from the selected hearing test and insert the extracted data into the current user hearing test at instances in which data is missing or sparse, thus “fleshing out” or “filling in” data-poor areas of the user's hearing test.

In the foregoing implementation, the method S110 can also test different frequency bands amongst a cohort of users—that is, a different set of frequency bands for each user in the cohort—to improve a global map of frequency-related hearing profiles. The method S110 can then interleave (e.g., combine, aggregate) test results in different frequency bands for multiple users yield more accurate hearing maps across the audible spectrum. For example, the method S110 can test three frequency bands for a first user, shift these three frequency bands upward by 500 HZ up for a second user, shift these three frequency bands downward by 500 HZ up for a third user, and so on to collect hearing data across the audible spectrum through a series of hearing tests completed by a variety of users.

The first method S100 can therefore collect and store hearing test results from multiple (e.g., thousands of) users, including multiple hearing tests from the same user (e.g., at different times, such as once per annum). Block S186 can further insert results from stored hearing tests into a new user hearing test to fill in data missing from the new hearing test as a result of the brevity of the new hearing test. In particular, the first method S100 can collect and store short (e.g., sixty-second) hearing tests that, independently, are error-prone and data-sparse but then combine the hearing test results from the multiple users to generate information-rich error-sparse hearing test data. Block S186 can then implement this information-rich error-sparse hearing test data to improve and even complete a new hearing test that is otherwise independently error-prone and data-sparse, such as by selecting stored hearing data to insert into the new hearing test based on a demographic of the corresponding user, a location of the user's hearing test, ambient noise data during the user's hearing test, etc. The first method S100 can also collect and store different types of hearing tests, such as short or superficial hearing tests engaged by new users, medium-length (e.g., five-minute) hearing tests engaged by experienced users, and long (e.g., one-hour) hearing tests engaged by high-need users (e.g., those with significant hearing loss), and Block S186 can cross-pollinate hearing tests of one type (e.g., short hearing tests) with hearing tests of another type(s) (e.g., medium-length and long hearing tests) to improve data density and/or accuracy of each new test.

Block S186 can similarly access results of other types of hearing tests, such as results of professional hearing tests implemented by physicians or audiologists. In this implementation, Block S186 can further normalize and/or reformat the results of the other types of hearing tests such that these results conform to a form or format of hearing tests collected within the corresponding system. Block S186 can then apply data from these other types of hearing tests to improve or complete the hearing test results for the user.

Once Block S186 improves or completes the hearing test results for the user, Block S170 can generate the baseline hearing profile for the user.

Additionally or alternatively, once Block S170 generates the baseline hearing profile for the user, Block S186 can compare the user's baseline hearing profile to stored hearing profiles of other users based on a demographic similarity between the user and the other users. Block S186 can further access one or more demographic, local noise, and/or location data of the user and compare these data to hearing profiles stored in the database, which may be tagged with similar demographic, local noise, and/or location data. Once Block S186 selects a previous hearing profile or other hearing-related data pertinent to the current user hearing profile, Block S186 can extract data from the selected previous hearing profile and insert the extracted data into the current user hearing profile at instances in which data is missing or sparse in the current user hearing profile, thus “fleshing out” or “filling in” data-poor areas of the user's hearing profile. Block S186 can implemented similar methods to compare the user's baseline hearing profile to stored hearing profiles of other users based on a similarities between hearing test results of the user and other users.

The first method S100 can therefore collect and store hearing profiles from multiple (e.g., thousands of) users, including multiple hearing profiles from the same user. Block S186 can thus insert data from stored hearing profiles into a new user hearing profile to fill in data missing from the new hearing profile, thereby improving the richness, resolution, and/or accuracy of the new hearing profile output in Block S170 despite an otherwise limited initial hearing dataset for the user.

However, Block S186 can function in any other way and implement any other machine learning, pattern matching, data source comparison technique, etc. to identify relevant stored hearing tests and/or stored hearing profiles to insert relevant data into and thus bolster a hearing test and/or a hearing profile of a user.

### 2. Second Method

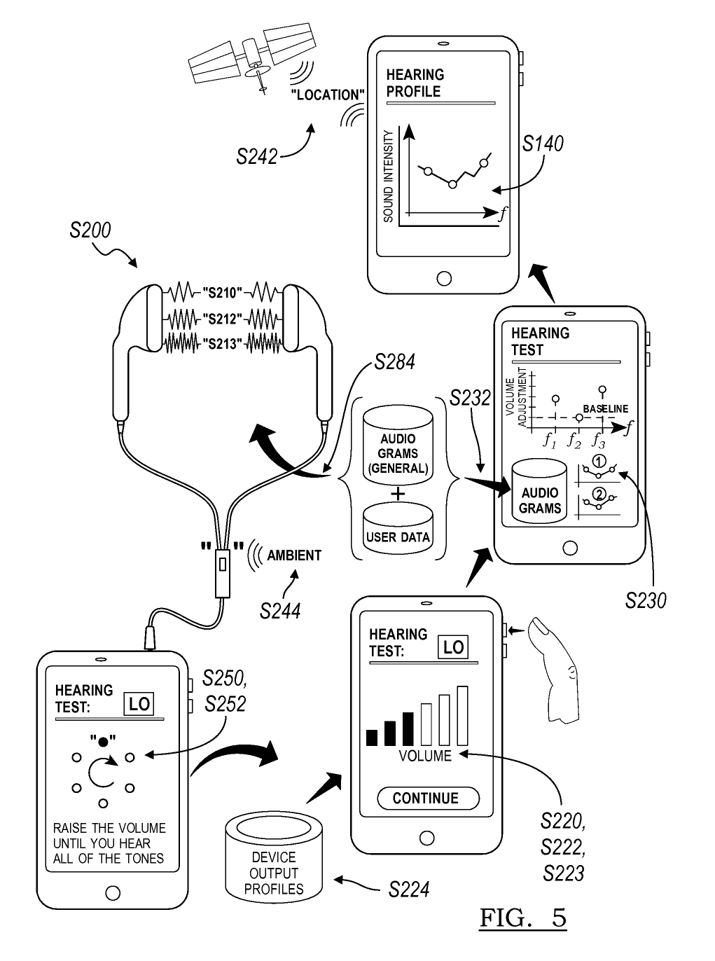

As shown in FIG. 5, a method for testing a hearing ability of a user includes: outputting a first audible tone including a first frequency in Block S210; recording a first volume adjustment for the first audible tone by the user in Block S220; outputting a second audible tone including a second frequency in Block S212; recording a second volume adjustment for the second audible tone by the user in Block S222; selecting a particular hearing model from a set of hearing models based on a difference between the first volume adjustment and the second volume adjustment in Block S230, each hearing model in the set of hearing models including a hearing test result corresponding to a previous patient; and generating a hearing profile for the user based on the particular hearing model result in Block S240.

One variation of the second method S200 for testing a hearing ability of a user includes: outputting a first set of distinct audible tones in a first sequence in Block S210, each audible tone in the first set of audible tones including a dominant frequency in a first audible frequency range; rendering a first visual cue corresponding to the first sequence in Block S250; recording a first volume adjustment for the first set of audible tones by the user in Block S220; outputting a second set of distinct audible tones in a second sequence in Block S212, each audible tone in the second set of audible tones including a dominant frequency in a second audible frequency range distinct from the first audible frequency range; rendering a second visual cue corresponding to the second sequence in Block S252; recording a second volume adjustment for the second set of audible tones by the user in Block S222; and generating a hearing profile for the user based on the first volume adjustment and the second volume adjustment in Block S240.

Generally, the second method S200 functions to generate a hearing profile that characterizes a user's hearing ability by testing the user's ability to hear a select subset of frequencies in the audible range, selecting a particular hearing test result from another user (or patient) who exhibits substantially similar hearing abilities at the select subset of frequencies, and applying data from the particular hearing test result to the user to fill in gaps in the hearing test at untested frequencies. In particular, the second method S200 functions to collect a limited amount of hearing ability data from a user within a limited period of time (e.g., thirty seconds), to characterize the limited amount of user hearing data, and to “flesh out” or complete an image of the user's hearing ability across (at least a portion of) the audible range by applying preexisting hearing ability data from one or more other users to the user based on the characterization of the limited amount of hearing data from the user. For example, the second method S200 can match volume adjustments entered by the user across two or more frequencies to hearing abilities at similar frequencies captured in an audiogram of a previous patient and then apply the audiogram of the previous patient to the user to estimate or predict the user's hearing abilities at other frequencies in the audible range. In another example, the second method S200 can transform volume adjustments entered by the user across two or more frequencies into a parametric hearing model to output a synthetic audiogram for the user, wherein the parametric hearing model is generated from a series of audiograms of various other patients such that the synthetic audiogram specific to the user is a composite of multiple audiograms of other patients. However, the second method S200 can apply hearing data from other patients to a (new) user in any other way to estimate or predict hearing abilities of the user across an audible range given a limited amount of user hearing data captured in a limited amount of time (e.g., less time than required to capture a full audiogram).

Like the first method S100, Blocks of the second method S200 can be implemented on or in conjunction with an audio output device, a computing device connected to an audio output device, and/or on a remote computer network in communication with the audio output device and/or the computing device. For example, Blocks S210, S212, S220, S222, S250, and S252 can be implemented on a user's mobile computing device (e.g., a smartphone, a tablet) outputting sound through a set of headphones (e.g., earbuds, over-ear headphones), and Blocks S230 and S240 can be implemented on a cloud-based computer system (e.g., Amazon EC2), which can transmit a generated hearing profile for the user back to the user's mobile computing device for subsequent application in adjustment of an audio engine parameter on the mobile computing device or on the headphones. However, Blocks of the second method S200 can be implemented by any other audio output device, computing device, and/or network, etc. to generate (and implement) a hearing profile for a user.

### 2.1 Audible Tones

Block S210 of the second method S200 recites outputting a first audible tone including a first frequency. Block S210 can also recite outputting a first set of distinct audible tones in a first sequence in Block S210, each audible tone in the first set of audible tones including a dominant frequency in a first audible frequency range. Block S212 of the second method S200 similarly recites outputting a second audible tone including a second frequency. Block S212 can also recite outputting a second set of distinct audible tones in a second sequence, each audible tone in the second set of audible tones including a dominant frequency in a second audible frequency range distinct from the first audible frequency range. As shown in FIG. 5, one variation of the second method S200 further includes Block S213, which similarly recites outputting a third audible tone including a third frequency.

Generally, Block S210 (and Blocks S212, 213, etc.) functions to output an audible sound (i.e., a tone or set of tones in the audible frequency range) for the user such that, when the user subsequently enters a volume adjustment for the audible sound, a subsequent Block of the second method S200 can correlate this volume adjustment with the user's ability to hear at one or more frequencies represented in the tone. In one implementation, as described above, Block S210 can execute on a computing device (e.g., a smartphone, a tablet, a laptop or desktop computer, a home or vehicle stereo system, etc.) to output an electronic signal corresponding to the first audible tone, such as through an audio jack, and an audio output device (e.g., a speaker, a pair of headphones) integrated into or connected to the computing device can convert the electronic signal into an audible signal for consumption by the user.

Blocks S210, S212, S213, etc. output different tones of different frequencies (or combinations of frequencies at different amplitudes) to prompt user responses to the different tones, which can be correlated with the user's ability to hear the different frequencies (or combinations of frequencies at different amplitudes) to subsequently generate the hearing profile for the user. For example, Block S210 can output a tone in a low-frequency band, Block S212 can output a tone in a mid-frequency band, and Block S213 can output a tone in a high-frequency band. Similarly, Block S210 can output a tone with a dominant frequency in the low-frequency band, and Blocks S212 and S213 can similarly output tones with dominant frequencies in the mid- and high-frequency bands, respectively. Thus, Blocks S210, S212, and S213 can prompt the user to provide hearing ability feedback at three distinct frequencies (or three predominant frequencies), thereby collecting relevant user data across the audible spectrum with a limited number of test points (i.e., frequencies). Generally, the second method S200 implements Blocks S210, S212, S213, etc. separately and sequentially as the user provides each volume adjustment corresponding to a previous output tone. In particular, once Block S220 records a first volume adjustment entered by the user in response to output of the first tone in Block S210, Block S212 outputs the second tone, and, once Block S222 records a second volume adjustment entered by the user in response to output of the second tone in Block S212, Block S213 outputs the third tone. Thus, Block S220, S222, etc. can thus collect—from the user—independent volume responses to different output audible tones.

In one implementation, Block S210 outputs the first audible tone of a first timbre, and Block S212 outputs the second audible tone of a second timbre. For example, in this implementation, Block S210 can output a recording of a low-E note or chord plucked or bowed on a three-quarter bass f, Block S212 can output a recording of a low-E note or chord plucked or bowed on a violin, and Block S213 can output a recording of a low-E note played on a piccolo. Blocks S210, S212, etc. can similarly output recordings of full musical chords, segments of songs, voices, or other sounds with predominant frequencies in corresponding sub-ranges within the audible range.

Alternatively, each of Blocks S210, S212, S213, etc. can output audible tones of singular frequencies. For example, Block S210 can output a single tone at 250 Hz, Block S212 can output a single tone at 1000 Hz, and Block S213 can output a single tone at 5000 Hz. Similarly, Blocks S210, S212, S213, etc. can output composite audible tones of select frequencies. For example, Block S210 can output a tone including a 245 Hz component, a 250 Hz component, and a 255 Hz component, all at substantially identical amplitudes. In this example, Block S212 can output a tone including a 990 Hz component, a 1000 Hz component, and a 1010 Hz component, all at substantially identical amplitudes. However, Blocks S210, S212, etc. can output tones, notes, recordings, or other sounds of any other form or type.

Blocks S210, S212, etc. can further output sets of tones within their respective sub-ranges in the audible range. In one implementation, Block S210 sequentially outputs a first set of tones within a first audible range that includes the first frequency, and Block S212 sequentially outputs a second set of tones within a second audible range including the second frequency, wherein the first audible range is discrete from the second audible range. In this implementation, Block S210 can serially and cyclically outputting a tone at a first unique frequency in the first audible frequency range, then a tone at a second unique frequency in the first audible frequency range, then a tone at a third unique frequency in the first audible frequency range. For example, Block S210 can output the first tone at a frequency of 245 Hz for a duration of two seconds followed by a 500 ms period silence, then output another tone at a frequency of 250 Hz for a duration of two seconds followed by a 500 ms period silence, and then output yet another tone at a frequency of 255 Hz for a duration of two seconds followed by a 500 ms period silence before repeating the sequence again. Block S210 can also arrange tones to create a musical effect during the hearing test. Block S210 can similarly output digital or recorded notes, chords, segments of songs, voices, or other sounds with predominant frequencies cyclically and in series. For example, Block S210 can output a recording of a plucked low-E note from a three-quarter bass, then output a recording of a plucked low-F note from the three-quarter bass, output a recording of a plucked low-G# note from a three-quarter bass, and then output a recording of a plucked low-G note from the three-quarter bass before repeating the sequence again. In these examples, Blocks S212, S213, etc. can similarly output a set of tones cyclically in series. Thus, by outputting a set of (nearby) tones within a frequency range in Block S210, S212, S213, etc., the second method S200 can substantially ensure that the user can respond to tests within each audible sub-range even if a particular frequency output in Blocks S210, S212, S213, etc. is not available (i.e., cannot be heard by) the user. However, Block S210, S212, S213, etc. can function in any other way to output one or more tones of any other type in any other suitable pattern or series.

As shown in FIG. 5, one variation of the second method S200 includes Block S284, which recites retrieving a demographic of the user from a computer network system and selecting the first set of distinct audible tones and the second set of distinct audible tones based on the demographic of the user. Generally, Block S284 implements methods and/or techniques of Block S184 described above to select particular tones, frequencies, and/or frequencies ranges to test in Block S210, S212, and/or S213, etc. For example, Block S284 can extract an age, gender, location, ethnicity, and/or occupation of the user from a social networking system, access hearing test results of other users or patients of sharing one or more demographic with the user, and adjust hearing test parameters according to trends in hearing tests of the other users. In this example, once demographic data is collected for the user, Block S284 can further access sets of audio tones from a database, each audio tone set associated with a different demographic (e.g., age group and/or occupation), and Block S284 can then match the user to a one or more particular audio tone sets in the database. Blocks S210, S212, and S213, etc. can then implement a selected audio tone set accordingly.

In the foregoing variation, various demographics can be correlated with different hearing abilities that can be leveraged with assigned audio tones (or audio tone sets) for testing Block S210, S212, etc. to prompt user volume adjustments that substantially adequately characterize the user's hearing ability across the audible range with only a modicum of tested frequencies. For example, machinists of twenty of more years may commonly experience significant hearing loss above 4000 Hz, while soldiers with combat experience may exhibit significant hearing loss below 200 Hz and moderate hearing loss between 200 and 800 Hz. In this example, Block S284 can specify audible tones in the 100-200 Hz range, 600-1200 Hz range, and 2000-3000 Hz range for a machinist in Blocks S210, S212, and S213, respectively, and Block S284 can specify audible tones in the 300-350 Hz range, 1400-1800 Hz range, and 4000-5000 Hz range for a soldier in Blocks S210, S212, and S213, respectively. Similarly, men of age sixty may commonly exhibit significant hearing loss above 4000 Hz compared to men of age sixteen, while women of age sixty may commonly exhibit significant hearing loss above 6000 Hz compared to women of age sixteen. In this example, Block S284 can specify audible tones of the 100 Hz, 600 Hz, and 2000 Hz for a man of age sixty, 600 Hz, 1500 Hz, and 5000 Hz range for a woman of age sixty, and 100 Hz, 1000 Hz, and 5000 Hz for a men and woman of age sixteen in Blocks S210, S212, and S213, respectively. Thus patterns in hearing loss and/or hearing ability can thus be characterized according to one or more such demographics or user characteristics, and Block S284 can select tones, frequencies, or dominant frequencies to output in Blocks S210, S212, etc. to match a hearing test to a demographic of the user.

Additionally or alternatively, Block S284 can select tones, frequencies, or dominant frequencies to output in Blocks S210, S212, etc. to support collection of additional hearing ability data for users of a certain demographic. For example, if Block S284 determines that the user is a male of age thirty who presently works in a law firm but toured with a punk band between the ages of seventeen and twenty-two but hearing tones at 350 Hz, 980 Hz, and 3345 Hz have not been tested for this demographic, Block S284 can thus select 350 Hz, 980 Hz, and 3345 Hz as test tone frequencies for Blocks S210, S212, and S213, respectively, accordingly. However, Block S284 can collect user demographic data in any other way and implement user demographic data according to any other schema to customize a tested tone or tested set of tones for the user.

### 2.2 Visual Cues

As shown in FIG. 5, one variation of the second method S200 includes Block S250, which recites rendering a first visual cue corresponding to the first sequence in Block S250. As shown in FIG. 5, this variation can also include Block S252, which recites rendering a second visual cue corresponding to the second sequence. Generally, Blocks S250, S252, etc. can function like Blocks S140A and S140B described above to render visual content on a display of a connected device (e.g., a smartphone, a tablet) to indicate to the user that the audio output device is currently outputting an audio signal. In particular, Block S250 can visually communicate to the user that audio is currently in playback even if the user cannot hear the audio, thus prompting the user to adjust a volume setting of the audio output device or the connected device up to a level that the user can audibly discern. Block S220 can subsequently capture this final volume setting for the first audible tone and/or the first set of audible tones to support selection of a particular hearing model in Block S230 and generation of a hearing profile for the user in Block S240.

In one implementation, Block S210 outputs the first audible tone at an initial volume below a standard minimum hearing threshold for the first frequency, and Block S250 displays a first visual cue of the first audible tone in playback substantially simultaneously with output of the first audible tone. For example, in the implementation described above in which Block S210 cyclically and sequentially outputs a set of audible tones within a sub-range of the audible range, Block S250 can render—on a display of the user's mobile computing device—a set of graphics, each graphic in the set representing one of the audible tones in the set of audible tones. In this example, as each subsequent audible tone in the set of audible tones is output in Block S210, Block S250 can pulse, change the color of, or otherwise visually alter each corresponding graphic, as shown in FIG. 5. Thus, even if the user cannot audibly discern an output sound, the user can identify a visual cue corresponding to an audible sound to confirm that a test is underway, which can function as a prompt to stimulate the user in entering a volume adjustment until one or all tones in the set of audible tones is heard by the user. As the second method S200 transitions to playback of the second audible tone or the second set of audible tones in Block S212, Block S250 can update the set of graphics according to the second frequency sub-range, such as by selecting a new size, shape, color, orientation, etc. of displayed graphics correlating to frequencies in the second sub-range. However, Block S250 can function in any other way to provide substantially real-time visual feedback corresponding to playback of audible tones in Blocks S210, S212, etc.

### 2.3 Volume Adjustment

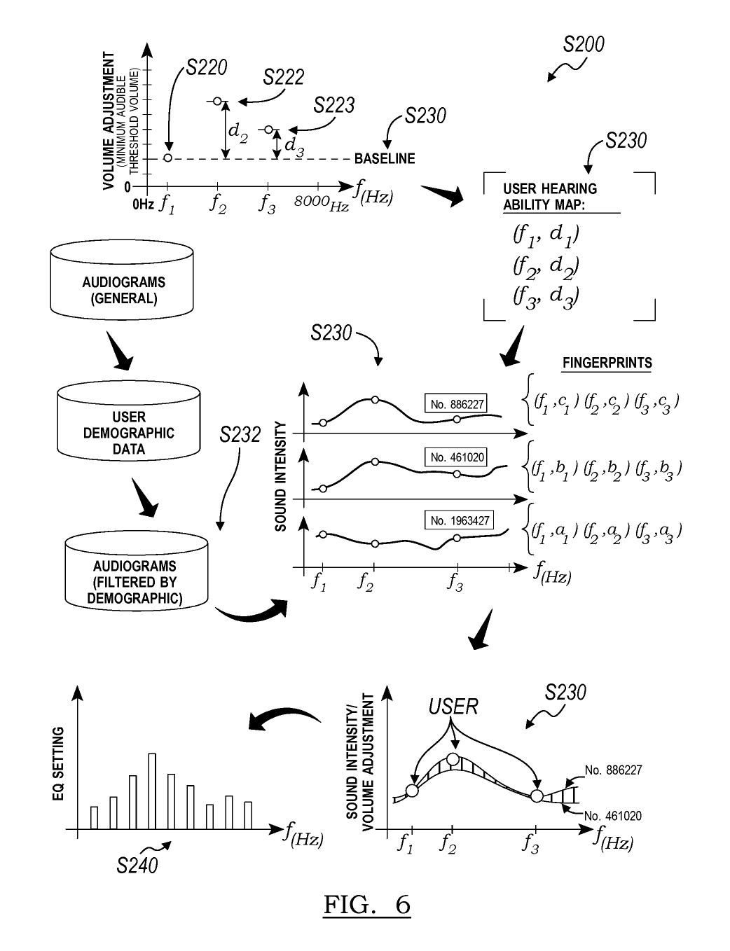

Block S220 of the second method S200 recites recording a first volume adjustment for the first audible tone (or for the first set of audible tones) by the user. Block S222 of the second method S200 similarly recites recording a second volume adjustment for the second audible tone (or for the second set of audible tones) by the user. As shown in FIG. 5, one variation of the second method S200 can also include Block S223, which similarly recites recording a third volume adjustment for the third audible tone by the user. Generally, Block S220, S222, S223, etc. function to record volume adjustments made by the user during playback of the first audible tone, the second audible tone, the third audible tone, respectively, etc., which can be correlated the use's ability to hear (i.e., audibly discern) corresponding frequencies in the audible range, as shown in FIG. 6.

In one implementation, Block S210 outputs the first audible initially at a minimum or “0” volume setting (i.e., at an inaudible or “0” volume level), and Block S250 displays a command on the user's mobile computing device to increase the volume setting of the mobile computing device (or the native application executing the second method S200) until the first audible tone (or all tones in the set of audible tones in the first frequency sub-range) is heard. Thus, as the user increases the volume setting of the mobile computing device, Block S220 can record the final volume adjustment set by the user. In one example, Block S250 can also prompt the user to confirm a user-set volume setting, such as by selecting a “Next” button rendered on the display, and Block S220 can capture the current volume setting when the “Next” button is selected by the user. Block S220 can thus store this volume setting for the first audible tone as a first volume adjustment, which can indicate a minimum audible threshold volume of the first frequency for the user. Block S220 can also calculate and store a difference between the initial volume of the first audible tone and the final volume adjustment entered by the user, such as in decibel change or absolute or relative (e.g., percentage) increase in peak, average, or continuous power output to drive the audio output device during playback of the first audible tone.

Blocks S222, S223, etc. can function similarly to capture volume settings entered by the user during a hearing test. Block S222 can also adjust a scale or granularity of a volume control provided to the user for entry of the second audio adjustment based on the first audio output adjustment entered by the user. Block S223 can similarly adjust a scale or granularity of the volume control provided to the user for entry of the third audio adjustment based on the second audio output adjustment entered by the user, and so on. Blocks S220, S222, and/or S223, etc. can also adjust the scale or granularity of the volume control provided to the user for entry of respective audio adjustments based on the audio output profile of the audio output device. However, Blocks S220, S222, S223, etc. can function in any other way to collect and record volume adjustments made by the user during playback of the first, second, and third audible tones, respectively.

As shown in FIG. 5, one variation of the second method S200 includes Block S224, which recites retrieving an output profile of an audio output device outputting the first audible tone. Generally, Block S224 can implement methods, techniques etc. described above in Block S110 to identify the audio output device (e.g., headphones) and/or the connected computing device (e.g., smartphone). For example, Block S224 can retrieve or wirelessly download a unique identifier (e.g., a serial number) from the audio output device and/or the connected computing device, or Block S224 can prompt the user to enter a make and/or model of the audio output device and/or the connected computing device. Alternatively, Block S224 can scan a barcode or implement machine vision techniques to identify the audio output device in a digital photographic image. However, Block S224 can collect identification information for the audio output device and/or the connected computing device in any other suitable way. Furthermore, once select identification information is collected, Block S224 can retrieve an audio profile for the device(s) from a database stored on the computing device or from a remote database (e.g., on a remote server and/or computer network).

Once an output profile of a related audio output device is selected in Block S224, Block S220 can normalize a volume adjustment entered by the user according to the output profile of the audio output device. In particular, Block S220 can normalize the first volume adjustment to a standard volume that is substantially consistent across a set of computing devices and/or audio output devices, etc. Blocks S222, S223, etc. can implement similar methods or techniques to normalize volume settings entered by the user for the second volume adjustment, the third volume adjustment, etc.

### 2.4 Hearing Model

Block S230 of the second method S200 recites selecting a particular hearing model from a set of hearing models based on a difference between the first volume adjustment and the second volume adjustment, each hearing model in the set of hearing models including a hearing test result corresponding to a previous patient. Generally, Block S230 functions to assign a hearing model to the user based on the first volume adjustment, the second volume adjustment, and/or the third volume adjustment, etc. The hearing tests of the previous users can be sourced from previous hearing tests completed as described herein, made publicly available by a government or regulatory agency, and/or collected by a third party, such as a health clinic, an audiologists, etc.

In one implementation, Block S230 selects the minimum or lowest volume adjustment—from a set of volume adjustments entered by the user for corresponding tested sub-ranges of the audible range—as a baseline volume adjustment setting, as shown in FIG. 6. Block S230 then subtracts the baseline volume adjustment setting from remaining volume adjustments, thereby normalizing the volume adjustments for a sound profile of the audio output device and/or the connected computing device. Block S230 subsequently characterizes the user's hearing ability across the range of audible frequencies based on normalized volume adjustments for particular corresponding frequencies. For example, Block S230 can generate a set of (multi-dimensional) data points that characterize the user's hearing ability, including a first data point specifying the first frequency of the first tone and a volume adjustment difference (e.g., in decibels, in volume adjustment increments, etc.) from the baseline volume adjustment, a second data point specifying the second frequency of the second tone and a volume adjustment difference from the baseline volume adjustment, and/or a third data point specifying the third frequency of the third tone and a volume adjustment difference from the baseline volume adjustment, etc. Block S230 can similarly generate data points specifying a predominant frequency in, a frequency sub-range including, etc. of the first tone or a center or average frequency from a set of frequencies output in Blocks S210, S212, S213, etc., which can be paired with a relative volume adjustment difference (i.e., relative to the baseline volume adjustment).

These discrete data points can thus define a hearing ability map that characterizes the user's hearing ability across the audible spectrum, as shown in FIG. 6. Block S230 can generate any number of these data points, such as one such data point for each audible tone or audible sub-range tested in Blocks S210, S212, S213, etc., and each data point can specify a frequency and a volume adjustment that are absolute or relative to any other data point in the set. However, Block S230 can function in any other way to generate a map characterizing the user's hearing ability across the audible spectrum from any number of discrete tested frequencies or frequency ranges.

Once a map of the data points of the user's hearing ability is generated, Block S230 can implement non-parametric methods or cooperate with Block S240 to implement parametric methods to assign a hearing model to the user. In one implementation, Block S230 accesses a set of previous hearing tests and compares the data points map of the user's hearing ability to a the previous hearing tests in the set. For example, each previous hearing test in the set can include an audiogram of a patient, each audiogram defining a hearing ability of the corresponding patient in the form of sound intensity (e.g., measured in decibels) versus frequency (i.e., in Hertz) for across frequencies in the audible range, in a sub-range of the audible range including fundamental frequencies of speech, and/or in a sub-range of the audible range including fundamental frequencies of music, etc. In particular, Block S230 can access from a database or generate automatically a fingerprint for each audiograms in the set, wherein a fingerprint of an audiogram specifies an absolute or relative sound intensity for each frequency tested in Blocks S210, S212, S213, etc. For example, Block S230 can generate (or access) a fingerprint for an audiogram that defines a baseline sound intensity as the sound intensity in the audiogram at a frequency corresponding to the frequency of the baseline volume adjustment setting in the user's hearing ability map. In this example, the fingerprint of the audiogram can further specify sound intensities relative to the baseline sound intensity at the remaining frequencies tested in Blocks S210, S212, S213, etc.

Block S230 can thus compare the user's hearing ability map to audiogram fingerprints to substantially match absolute or relative volume adjustments by the user to absolute or relative sound intensities in a single audiogram (or a set of audiograms relevant to the user and averaged or otherwise combined according to one or more trends into a single composite audiogram) at the frequencies tested in Blocks S210, S212, S213, etc., as shown in FIG. 6. In particular, a volume adjustment set by the user and normalized for the baseline volume adjustment setting can define a minimum audible threshold volume at a corresponding frequency for the user, and a normalized sound intensity defined in the audiogram can similarly define a define minimum audible threshold volume at a corresponding frequency for the patient. Block S230 can therefore select a particular audiogram—from the set of audiograms—that defines minimum audible threshold volumes of a previous patient that best match minimum audible threshold volumes for the user tested at select frequencies in Blocks S210, S212, S213, etc. Block S230 can then pass this particular audiogram, including the sound intensities relative to frequencies for the corresponding patient, to Block S240 for implementation in generating the hearing profile for the user.

In a similarly implementation, Block S230 matches the user's hearing ability map to a composite hearing test including data from multiple previous hearing tests (e.g., audiograms, articulation indices, minimum audibility curves, and/or equal-loudness contours, etc.). For example, each composite hearing test can include aggregate data specifying a relationship between frequency and hearing ability or perception within a group of patients with similar hearing abilities such that a composite hearing test defines an average hearing ability or a range of hearing abilities (e.g., minimum sound intensities) across the audible, speech, music, or other frequency range. Block S230 can thus implement similar methods or techniques to match the user's hearing ability map to a previous hearing test that includes hearing data from multiple previous patients or users. For example, Block S230 can select a particular composite (e.g., synthetic) audiogram from a set of composite audiograms based on the difference between the first volume adjustment and the second volume adjustment entered by the user in Block S220 and S222, wherein each composite audiogram includes data from multiple preexisting audiograms corresponding to a various previous patients.

Alternatively, Block S230 can pass data points from the user's hearing ability map directly to Block S240, wherein Block S240 inserts these data points into a parametric hearing model to generate the user's hearing profile. For example, the second method S200 can generate and/or access a parametric hearing model based on real hearing test data from a variety of previous patients. In this example, the second method S200 can implement machine learning to update and improve the parametric hearing model as new audiograms or other “professional” or “medical-grade” hearing test data is collected and made available. Block S240 can then insert the user's (normalized) minimum audible threshold volume at select (e.g., three) frequencies into the parametric hearing model to generate a hearing profile that “fills in” the user's hearing ability across other frequencies in the audible range. However, Block S230 and Block S240 can cooperate in any other way to generate a hearing profile for the user based on a limited set of user hearing data defining relative audible threshold volumes of the user at select frequencies in the audible range.

However, Block S230 can select a particular hearing model for the user in any other way and based on an audiogram, an articulation index, a minimum audibility curve, an equal-loudness contour, or any other hearing data collected from a previous patient or user in any other suitable way.

As shown in FIG. 5, one variation of the second method S200 includes Block S232, which recites receiving a demographic of the user and implementing the demographic of the user to populate the set of hearing models from a database of preexisting hearing test results based on a similarity between the demographic of the user and demographic information of patients corresponding to preexisting hearing test results in the database of preexisting hearing test results. Generally, Block S232 can implement methods or techniques of Blocks S184 and S284 described above to collect user demographic information, such as age, gender, location, ethnicity, and/or occupation, and Block S232 subsequently implements this user demographic data to filter available previous hearing tests, composite hearing tests, parametric hearing models, etc. particular to the user.

In one implementation, Block S232 implements demographic data of the user to identify previous audio test results that do not align with one or more demographics of the user and then discards these previous audio test results from comparison with the user's hearing ability map. For example, Block S232 can filter out all previous audio test results from patients of a different age group, ethnicity, and/or gender, etc. such that the user's hearing ability map is only compared to hearing data from similar patients. In a similar implementation, Block S232 implements demographic data of the user to identify previous audio test results that do not align with one or more demographics of the user and then discards these previous audio test results generation of composite hearing tests. For example, Block S232 can discard previous hearing tests from patients that are dissimilar in one or more demographics from the user, and Block S230 can group remaining hearing test results by similarities in indicated hearing profile across the audible range and generate composite hearing tests from the grouped hearing test results. In yet another implementation, Block S240 accesses and/or generates demographic-dependent parametric hearing models, and Block S232 implements the user's demographic data to discard substantially irrelevant parametric hearing models or to select a particular parametric hearing model for application in Block S240 to generate a hearing profile for the user. However, Block S232 can cooperate with Block S230 and/or Block S240 in any other way to implement user demographic data in any other way to select a particular hearing model or to implement a particular parametric hearing model to output a hearing profile for the user, respectively.

### 2.5 Hearing Profile

Block S240 of the second method S200 recites generating a hearing profile for the user based on the particular hearing model result. Generally, Block S240 functions to output a hearing profile for the user based on a particular hearing model selected in Block S230 and/or to generate a hearing profile for the user based on a parametric hearing model and a user hearing ability map output in Block S230. For example, Block S240 can cooperate with Blocks S210, S212, S220, S222, S230, etc. to output a series of audible tones, to collect volume adjustments entered by the user, assign a hearing model to the user, and to output a hearing profile for the user all in less than sixty seconds from output of the first audible tone. In particular, Block S240 can estimate (e.g., predict, project) the user's hearing ability across a range of frequencies in the audible range—such as within a sub-range of the audible range including fundamental frequencies of speech and/or fundamental frequencies of music—based on audible threshold volumes of the user collected at a limited number of (e.g., two or three) frequencies within the audible range.

In one implementation, Block S240 assigns a particular hearing test result (e.g., an audiogram) selected in Block S230 to the user as the user's hearing profile. For example, Block S240 can generate a hearing profile that projects a hearing ability of the user at frequencies spanning the audible spectrum based predominantly on a particular preexisting audiogram selected in Block S230. In a similar implementation, Block S240 can transform the particular hearing test selecting in Block S230 into an equalizer (EQ) setting or audio engine parameter for the corresponding audio output device, the connected computing device, and/or the combination of the audio output device with the connected computing device. For example, in this implementation, Block S240 can generate an audio engine parameter (e.g., an equalizer setting) that boosts frequencies projected as difficult for the user to hear (i.e., are associated with high audible threshold volumes) and that attenuates (or does not change) frequencies at which the user hears normally (e.g., frequencies at which the user does not exhibit substantive hearing loss).

In another implementation, Block S230 selects a set of particular hearing tests that substantially match the user's hearing ability map across the range of frequencies tested in Blocks S210, S212, S213, etc., and Block S240 averages or otherwise combines the set of particular hearing tests into a composite hearing test defining the hearing profile for the user. In a similar implementation, Block S230 selects a particular hearing test for each of a sub-range of frequencies tested in Blocks S210, S212, S213, etc., and Block S240 combines (e.g., arranges linearly according to frequency) the hearing tests across the sub-ranges into a composite hearing test defining the hearing profile for the user.

Yet alternatively, as described above, Block S240 can insert data from a user hearing ability map into a parametric hearing model to output the user hearing profile, such as in the form of projected hearing abilities (e.g., a minimum audible threshold volumes) across a range of frequencies or as an EQ setting that accommodates projected hearing abilities of the user across a range of frequencies, as shown in FIG. 6.

However, Block S240 can function in any other way to output a hearing profile of any other form for the user. The hearing profile can be subsequently implemented on any one or more audio output devices (e.g., headphones, a home or vehicle stereo system), computing devices (e.g., a smartphone, a tablet), and/or within a native application executing on a computing device (e.g., a native phone call application executing on a smartphone) to augment the user's listening experience and/or to enable the user to better discern audible sounds output from the audio output device and/or the computing device.

As shown in FIG. 5, one variation of the second method S200 includes Block S242, which recites retrieving a location of the mobile computing device, associating the hearing profile with the location, and adjusting an audio engine parameter of the mobile computing device based on the hearing profile in response to detection of the mobile computing device within a threshold range of the location. Generally, Block S242 can implement methods or techniques described above in Block S180 to determine a location of the user and then to index the current hearing profile of the user (e.g., in a database of hearing profiles for the user) according to the corresponding location. Thus, when the user returns to the location at a later time, the audio output device and/or the computing device can select and implement the hearing profile corresponding to the location. For example, when the user is within a threshold distance from a home location (e.g., within 100 meters of a stored home location), the computing device can retrieve a first hearing profile generated in the user's home and thus associated with the user's home, and when the user is within a threshold distance from a work location (e.g., within 50 meters of a stored office location), the computing device can retrieve a second hearing profile generated in the user's office and thus associated with the user's work location. In this example, the threshold distance from each hearing profile-related location—or range of applicability of a hearing profile across a location—can be fixed across all location types, location type-dependent (e.g., 100 meters for home locations, 50 meters for work locations), user-selected, etc.

As shown in FIG. 5, a similar variation of the second method S200 includes Block S244, which recites generating a characterization of ambient noise based on an output of a microphone of a connected device (e.g., a cellular phone). For example, Block S244 can implement methods or techniques similar to Block S182 described above to collect ambient noise data through a microphone in the connected device and/or in the audio output device and to characterize the collected ambient noise. For example, Block S244 can characterize loud and soft noises in the microphone output by frequency or frequency range and then assign this characterization to a particular hearing profile generated in Block S240. Thus, at a subsequent time after a particular hearing profile is generated for the user, when the user returns to a setting with ambient noise substantially matching the ambient noise characterization assigned to the particular hearing profile, the computing device and/or the audio output device can select the particular hearing profile from a set of hearing profiles generated for the user and then implement the particular hearing profile accordingly, such as until the user's location changes and/or until an ambient noise condition proximal the user changes. For example, Block S244 can characterize ambient noise proximal the user as a noisy restaurant before, during, or shortly after Block S240 outputs a first hearing profile, and Block S244 can associate this first hearing profile with a noisy restaurant environment. In this example, Block S244 can also characterize ambient noise proximal the user as a quiet office before, during, or shortly after Block S240 outputs a second hearing profile, and Block S244 can associate this first hearing profile with a quiet office environment. (Block S242 can also assign particular locations to these hearing profiles accordingly.) Thus, when the computing device and/or the audio output device subsequently characterizes ambient noise proximal the user as a noisy restaurant environment, the computing device can access and implement the first profile, and when the computing device and/or the audio output device subsequently characterizes ambient noise proximal the user as a quiet office environment, the computing device can access and implement the second profile. However, Block S244 can function in any other way to characterize and assign an ambient noise condition to a hearing profile output in Block S240.

The systems and methods of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, or any suitable combination thereof. Other systems and methods of the embodiments can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor, though any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

We claim:

1. A method for testing a hearing ability of a user, comprising:
outputting a first audible tone comprising a first frequency;
recording a first volume adjustment for the first audible tone by the user;
outputting a second audible tone comprising a second frequency;
recording a second volume adjustment for the second audible tone by the user;
selecting a particular hearing model from a set of hearing models based on a difference between the first volume adjustment and the second volume adjustment, each hearing model in the set of hearing models comprising a hearing test result corresponding to a previous patient; and
generating a hearing profile for the user based on the particular hearing model result.

2. The method of claim 1, further comprising outputting a third audible tone comprising a third frequency and recording a third volume adjustment for the third audible tone by the user, wherein selecting the particular hearing model comprises selecting the particular hearing model further based on a difference between the first volume adjustment and the third volume adjustment.

3. The method of claim 1, wherein recording the first volume adjustment for the first audible tone comprises storing the first volume adjustment as a minimum audible threshold of the first frequency for the user, wherein recording the second volume adjustment for the second audible tone comprises storing the second volume adjustment as a minimum audible threshold of the second frequency for the user, and wherein selecting the particular hearing model comprises matching a relative difference between the minimum audible threshold of the first frequency and the minimum audible threshold of the second frequency with a relative difference between audio thresholds defined in the particular hearing model for the first frequency and the second frequency.

4. The method of claim 1, wherein outputting the first audible tone comprises outputting the first audible tone at an initial volume below a standard minimum hearing threshold for the first frequency and displaying a first visual cue of the first audible tone substantially simultaneously.

5. The method of claim 4, wherein recording the first volume adjustment for the first audible tone comprises prompting the user to increase a volume output of the first audible tone until the first audible tone is audible, recording a final volume adjustment for the first audible tone selected by the user, and correlating a difference between the initial volume and the final volume adjustment with a relative hearing ability of the user at the first frequency.

6. The method of claim 1, wherein outputting the first audible tone comprises sequentially outputting a first set of tones within a first audible range comprising the first frequency, and wherein outputting the second audible tone comprises sequentially outputting a second set of tones within a second audible range comprising the second frequency, the first audible range distinct from the second audible range.

7. The method of claim 1, wherein outputting the first audible tone comprises outputting the first audible tone of a first timbre, and wherein outputting the second audible tone comprises outputting the second audible tone of a second timbre.

8. The method of claim 1, further comprising receiving a demographic of the user and populating the set of hearing models from a database of preexisting hearing test results based on a similarity between the demographic of the user and demographic information of patients corresponding to preexisting hearing test results in the database of preexisting hearing test results.

9. The method of claim 8, further comprising selecting the first audible tone and the second audible tone based on substantially unique frequency-dependent demographic characterizations of preexisting hearing test results in the database at the first frequency and at the second frequency.

10. The method of claim 8, wherein populating the set of hearing models comprises matching the demographic of the user to a demographic of a patient associated with a hearing test result in the database, the demographic comprising a location of the user.

11. The method of claim 8, wherein populating the set of hearing models comprises matching the demographic of the user to a demographic of a patient associated with a hearing test result in the database, the demographic comprising an age and a gender of the user.

12. The method of claim 1, wherein selecting the particular hearing model comprises selecting a particular preexisting audiogram from a set of preexisting audiograms, and wherein generating the hearing profile for the user comprises inserting, into the hearing profile of the user, hearing abilities defined in the particular preexisting audiogram at frequencies spanning the audible spectrum.

13. The method of claim 1, wherein selecting the particular hearing model comprises selecting a particular hearing model from a set of hearing models based on the difference between the first volume adjustment and the second volume adjustment, each hearing model in the set of hearing models comprising a composite of a plurality of preexisting audiograms corresponding to a plurality of previous patients.

14. The method of claim 1, further comprising retrieving an output profile of an audio output device outputting the first audible tone, wherein recording the first volume adjustment for the first audible tone comprises normalizing the first volume adjustment according to the output profile of the audio output device.

15. The method of claim 1, wherein outputting the first audible tone comprises outputting the first audible tone through a mobile computing device, and further comprising retrieving a location of the mobile computing device, associating the hearing profile with the location, and adjusting an audio engine parameter of the mobile computing device based on the hearing profile in response to detection of the mobile computing device within a threshold range of the location.

16. The method of claim 1, wherein outputting the first audible tone comprises outputting the first audible tone at an audio jack within a cellular phone, and further comprising generating a characterization of ambient noise based on an output of a microphone within the cellular phone, wherein generating the hearing profile comprises associating the hearing profile with the characterization of ambient noise.

17. The method of claim 1, wherein generating the hearing profile for the user comprises generating the hearing profile for the user within sixty seconds of outputting the first audible tone.

18. A method for testing a hearing ability of a user, comprising:
outputting a first set of distinct audible tones in a first sequence, each audible tone in the first set of audible tones comprising a dominant frequency in a first audible frequency range;
rendering a first visual cue corresponding to the first sequence;
recording a first volume adjustment for the first set of audible tones by the user;
outputting a second set of distinct audible tones in a second sequence, each audible tone in the second set of audible tones comprising a dominant frequency in a second audible frequency range distinct from the first audible frequency range;
rendering a second visual cue corresponding to the second sequence;
recording a second volume adjustment for the second set of audible tones by the user; and
generating a hearing profile for the user based on the first volume adjustment and the second volume adjustment.

19. The method of claim 18, wherein outputting the first set of distinct audible tones in a first sequence comprises serially and cyclically outputting a tone at a first unique frequency in the first audible frequency range, followed by a tone at a second unique frequency in the first audible frequency range, followed by a tone at a third unique frequency in the first audible frequency range.

20. The method of claim 18, further comprising retrieving a demographic of the user from a computer network system and selecting the first set of distinct audible tones and the second set of distinct audible tones based on the demographic of the user.

21. The method of claim 20, wherein selecting the first set of distinct audible tones and the second set of distinct audible tones based on the demographic of the user comprises selecting a predefined set of audio tone sets comprising the first set of distinct audible tones and the second set distinct audio tones, the predefined set of audio tone sets assigned to the demographic.

22. The method of claim 18, wherein generating the hearing profile comprises selecting a particular preexisting audiogram from a set of preexisting audiograms based on a difference between the first volume adjustment and the second volume adjustment relative to the first audible frequency range and the second audible frequency range, and wherein generating the hearing profile comprises generating the hearing profile based on the particular preexisting audiogram.

23. The method of claim 18, wherein outputting the first set of distinct audible tones comprises outputting the first set of distinct audible tones at an initial minimum volume setting on a mobile computing device, wherein rendering the first visual cue comprises rendering the first visual cue on a display of the mobile computing device.
